(19) United States
(12) Patent Application Publication
(10) Pub. No.: US 2016/0310501 A1
(43) Pub. Date: Oct. 27, 2016

(54) GABAPENTINOIDS AND SIGMA RECEPTOR LIGANDS COMBINATIONS

(71) Applicant: LABORATORIOS DEL DR. ESTEVE, S.A., Barcelona (ES)

(72) Inventors: Daniel ZAMANILLO-CASTANEDO, Barcelona (ES); Enrique PORTILLO-SALIDO, Barcelona (ES)

(21) Appl. No.: 15/104,752

(22) PCT Filed: Dec. 16, 2014
(86) PCT No.: PCT/EP2014/077992

§ 371 Date: Jun. 15, 2016

(30) Foreign Application Priority Data
Dec. 17, 2013 (EP) 13382517.4

(51) Int. Cl. A61K 31/5377; A61K 31/195

(52) U.S. Cl. CPC A61K 31/5377; A61K 31/195

(57) ABSTRACT

The invention refers to a synergistic combination comprising a Sigma ligand of general formula (I), and a Gabapentinoid, a medicament comprising said active substance combination, and the use of said active substance combination for the manufacture of a medicament, particularly for the prophylaxis and/or treatment of pain.

### FIELD OF THE INVENTION

The present invention relates to an active substance combination, pharmaceutical compositions containing it and their use in medicine, particularly for the prophylaxis and/or treatment of pain.

### BACKGROUND

The treatment of pain conditions is of great importance in medicine. There is currently a world-wide need for additional pain therapy. The pressing requirement for a specific treatment of pain conditions is documented in the large number of scientific works that have appeared recently in the field of applied analgesics.

PAIN is defined by the International Association for the Study of Pain (IASP) as “an unpleasant sensory and emotional experience associated with actual or potential tissue damage, or described in terms of such damage” (IASP, Classification of chronic pain, 2nd Edition, IASP Press (2002), 210). Although it is a complex process influenced by both physiological and psychological factors and is always subjective, its causes or syndromes can be classified. Pain can be classified based on temporal, aetiological or physiological criteria. When pain is classified by time, it can be acute or chronic. Aetiological classifications of pain are malignant or non-malignant. A third classification is physiological, which includes nociceptive pain (results from detection by specialized transducers in tissues attached to A-delta and C-fibers), that can be divided into somatic and visceral types of pain, and neuropathic pain (results from irritation or damage to the nervous system), that can be divided into peripheral and central neuropathic pain. Pain is a normal physiological reaction of the somatosensory system to noxious stimulation which alerts the individual to actual or potential tissue damage. It serves a protective function of informing us of injury or disease, and usually remits when healing is complete or the condition is cured. However, pain may result from a pathological state characterized by one or more of the following: pain in the absence of a noxious stimulus (spontaneous pain), increased duration of response to brief stimulation (ongoing pain or hyperpathia), reduced pain threshold (allodynia), increased responsiveness to suprathreshold stimulation (hyperalgesia), spread of pain and hyperalgesia to uninjured tissue (referred pain and secondary hyperalgesia), and abnormal sensations (e.g., dysesthesia, paresthesia).

Gabapentinoids such as gabapentin and pregabalin are anticonvulsants which have been widely used for pain control in different medical situations. These aminobutyric acid analogues have shown analgesic antinociceptive effects. Several hypotheses have been proposed for the gabapentinoids' analgesic mechanism of action. Gabapentin and pregabalin both bind to the α2δ-1 subunit of the voltage-dependent calcium channel, resulting in a reduction in the influx of calcium at nerve terminals (Field et al., 2006; Davies et al., 2007). In turn, this reduces the release of several neurotransmitters, including glutamate, noradrenaline, and substance. This mechanism has been consistently observed across a wide range of studies and probably accounts for the majority of analgesic activity that is seen using these compounds (Taylor et al., 2009).

Pregabalin was designed as a more potent successor to gabapentin. It is structurally related to gabapentin and the two drugs are largely indistinguishable in terms of their pharmacological profile. Pregabalin is an anticonvulsant drug used for neuropathic pain and as an adjunct therapy for partial seizures with or without secondary generalization in adults. It has also been found effective for generalized anxiety disorder and is approved for this use in the European Union and Russia.

Pregabalin is marketed by Pfizer under the trade name Lyrica. Pfizer described that the drug could be used to treat epilepsy, post-herpetic neuralgia, diabetic peripheral neuropathy and fibromyalgia.

The most common side effects of pregabalin and gabapentin are dizziness, drowsiness, dry mouth, edema (accumulation of fluid), blurred vision, weight gain, and difficulty concentrating. Other side effects include reduced blood platelet counts, and increased blood creatinine kinase levels. Increased creatinine kinase can be a sign of muscle injury, and in clinical trials three patients experienced rhabdomyolysis (severe muscle injury). Therefore, patients should report unexplained muscle pain, tenderness or weakness to their doctors, especially if associated with fever and malaise (reduced well-being). In addition, antiepileptic medications have been associated with increased risk of suicidal thinking and behavior. Anyone considering the use of antiepileptic drugs must balance this risk of suicide with the clinical need. Patients who are started on therapy should be closely observed for clinical worsening, suicidal thoughts, or unusual changes in behavior. Thus therapeutic utility of Gabapentinoids is limited by their undesirable adverse effects (Perret et al., 2009).

Two subtypes of Sigma receptors (Sigma-1 and Sigma-2 receptors) have been identified (Cobos et al., 2008). Confused with opioid receptors for many years due to the cross-reactivity of some ligands, the Sigma-1 receptor is a 24-kDa molecular mass protein of 223 amino acids anchored to the endoplasmic reticulum and plasma membranes (Cobos et al., 2008; Maurice and Su, 2009). Sigma-1 receptor is a unique ligand-regulated molecular chaperone which is activated under stress or pathological conditions and interacts with several neurotransmitter receptors and ion channels to modulate their function. The effects reported preclinically with Sigma-1 receptor ligands are consistent with a role for Sigma-1 receptor in central sensitization and pain hypersensitivity and suggest a potential therapeutic use of Sigma-1 receptor antagonists for the management of neuropathic pain as monotherapy (Romero et al., 2012).

Pyrazole derivatives of general formula (I) according to the present invention are described in WO 2006/021462 as compounds having pharmacological activity towards the sigma (o) receptor useful, inter alia, in the prophylaxis and/or treatment of pain.

Pharmaceutical compositions (WO 2011/064296 A1), salts (WO 2011/064315 A1), polymorphs and solvates (WO 2011/095579 A1), and other solid forms (WO 2012/019984 A1) of said sigma ligands of formula (I) have been also disclosed as well as combinations with other active substances such a with opioids or opiates (WO 2009/130310 A1, WO 2012/016980 A2, WO 2012/072782 A1) or with chemotherapeutic drugs (WO 2011/018487 A1, WO 2011/144721 A1).

As mentioned above, therapeutic utility of Gabapentinoids is limited by undesirable adverse effects including cardiovascular and gastrointestinal toxicity. Thus, strategies aimed to reduce doses needed for Gabapentinoids indications, especially for analgesia, are desirable in order to improve their therapeutic window and extend their use in clinics.

### BRIEF DESCRIPTION OF THE INVENTION

It is an object of the present invention to provide a medicament suitable for the prophylaxis and/or treatment of pain, which preferably does not show the undesired side effects of the Gabapentinoids when used for the prophylaxis and/or treatment of pain, or at least less frequent and/or less pronounced.

The inventors of the present invention have found and demonstrated that the administration of some specific Sigma receptor ligands in conjunction with Gabapentinoids surprisingly potentiates synergistically the analgesia.

In particular, the inventors of the present invention have found and demonstrated that the administration of some specific Sigma receptor ligands in conjunction with Gabapentinoids potentiates synergistically the analgesic effect of the latter, indicating that the combination of a Sigma ligand and a Gabapentinoid reduces the doses of the latter needed to obtain effective analgesia.

Likewise, the inventors of the present invention have found and demonstrated that the administration of some specific Sigma receptor ligands in conjunction with Gabapentinoids potentiates synergistically the analgesic effect of Sigma ligands.

Therefore, one aspect of the present invention relates to a synergistic combination comprising at least one Gabapentinoid selected from a α2δ subunit calcium channel ligand having an inhibition constant (Ki) of 1000 nM or less and at least one Sigma ligand of general formula (I)

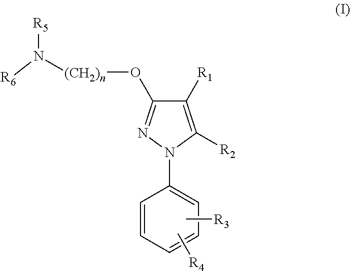

wherein,
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
R₂ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen, or together with the phenyl they form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
or together form, with the nitrogen atom to which they are attached, a substituted or unsubstituted, aromatic or non-aromatic heterocyclyl group;
n is selected from 1, 2, 3, 4, 5, 6, 7 and 8;
t is 0, 1 or 2;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen, or a pharmaceutically acceptable salt, isomer, prodrug or solvate thereof.

More preferably, the Sigma ligands according to the present invention are selective Sigma-1 antagonist receptor ligands of above defined general formula (I) or a pharmaceutically acceptable salt, isomer, prodrug or solvate thereof.

Another aspect of this invention refers to the synergistic combination comprising at least one Sigma ligand of general formula (I) as defined above, or a pharmaceutically acceptable salt, isomer, prodrug or solvate thereof, and at least one Gabapentinoid as defined above for use in medicine.

Another aspect of this invention refers to the synergistic combination comprising at least one Sigma ligand of general formula (I) as defined above, or a pharmaceutically acceptable salt, isomer, prodrug or solvate thereof, and at least one Gabapentinoid as defined above for use in the prophylaxis and/or treatment of pain.

Another aspect of this invention refers to the use of the synergistic combination comprising at least one Sigma ligand of general formula (I) as defined above, or a pharmaceutically acceptable salt, isomer, prodrug or solvate thereof, and at least one Gabapentinoid as defined above for manufacturing a medicament for the prophylaxis and/or treatment of pain.

Another aspect of the invention is a method of treatment and/or prophylaxis of a patient suffering from pain, or likely to suffer pain, the method comprising administering to the patient in need of such a treatment or prophylaxis a therapeutically effective amount of a synergistic combination comprising at least one Sigma ligand of general formula (I) as defined above, or a pharmaceutically acceptable salt, isomer, prodrug or solvate thereof, and at least one Gabapentinoid as defined above.

Another aspect of this invention refers to the synergistic combination comprising at least one Sigma ligand of general formula (I) as defined above, or a pharmaceutically acceptable salt, isomer, prodrug or solvate thereof, and at least one Gabapentinoid as defined above for use in the prophylaxis and/or treatment of pain by potentiating the analgesic effect of the Gabapentinoid.

Another aspect of this invention refers to the use of the synergistic combination comprising at least one Sigma ligand of general formula (I) as defined above, or a pharmaceutically acceptable salt, isomer, prodrug or solvate thereof, and at least one Gabapentinoid as defined above for manufacturing a medicament for the prophylaxis and/or treatment of pain by potentiating the analgesic effect of the Gabapentinoid.

Another aspect of this invention refers to the use of Sigma ligands of general formula (I) for potentiating the analgesic effect of Gabapentinoids as defined above.

The pharmaceutical synergistic combination of the invention may be formulated for its simultaneous, separate or sequential administration.

These aspects and preferred embodiments thereof are additionally also defined hereinafter in the detailed description, as well as in the claims.

### BRIEF DESCRIPTION OF THE FIGURES

FIG. 1: Potentiation of pregabalin analgesia (0.04 mg/kg) by compound 63.HCl (5, 10, 20, 40 and 80 mg/kg) in the mechanical allodynia of the post-operative pain model in rats (n=10), *: p<0.05; ns: p>0.05 Dunnett, compound 63.HCl+pregabalin vs. pregabalin.

FIG. 2: Potentiation of a subactive dose of gabapentin (10 mg/kg) by compound 63.HCl (10, 20, 40 and 80 mg/kg) in the mechanical allodynia of the post-operative pain model in rats (n=10), *: p<0.05; ns:p>0.05 Dunnett, compound 63.HCl+gabapentin vs. gabapentin.

### DETAILED DESCRIPTION OF THE INVENTION

The efficacy of the active components can sometimes be improved by addition of other (active) ingredients. More rarely, the observed efficacy of the combination of ingredients can be significantly higher than what would be expected from the amounts of the individual ingredients used, thus indicating potentiation of the activity of the components of the combination.

The present inventors have found that Sigma receptor ligands of general formula (I) are able to potentiate the analgesic effect of Gabapentinoids.

In the context of the present invention, the following terms have the meaning detailed below.

“Alkyl” refers to a straight or branched hydrocarbon chain radical containing no unsaturation, and which is attached to the rest of the molecule by a single bond. Typical alkyl groups have from 1 to about 12, 1 to about 8, or 1 to about 6 carbon atoms, e.g., methyl, ethyl, n-propyl, i-propyl, n-butyl, t-butyl, n-pentyl, etc. Alkyl radicals may be optionally substituted by one or more substituents such as aryl, halo, hydroxy, alkoxy, carboxy, cyano, carbonyl, acyl, alkoxycarbonyl, heterocyclyl, amino, nitro, mercapto, alkylthio, etc. If substituted by aryl, it corresponds to an “arylalkyl” radical, such as benzyl or phenethyl. If substituted by heterocyclyl, it corresponds to a “heterocyclylalkyl” radical.

“Alkenyl” refers to a straight or branched hydrocarbon chain radical containing at least two carbon atoms and at least one unsaturation, and which is attached to the rest of the molecule by a single bond. Typical alkenyl radicals have from 2 to about 12, 2 to about 8 or 2 to about 6 carbon atoms. In a particular embodiment, the alkenyl group is vinyl, 1-methyl-ethenyl, 1-propenyl, 2-propenyl, or butenyl.

“Alkynyl” refers to a straight or branched hydrocarbon chain radical containing at least two carbon atoms and at least one carbon-carbon triple bond, and which is attached to the rest of the molecule by a single bond. Typical alkynyl radicals have from 2 to about 12, 2 to about 8 or 2 to about 6 carbon atoms. In a particular embodiment, the alkynyl group is ethynyl, propynyl (e.g. 1-propynyl, 2-propynyl), or butynyl (e.g. 1-butynyl, 2-butynyl, 3-butynyl).

“Cycloalkyl” refers to an alicyclic hydrocarbon which is saturated or partially saturated. Typical cycloalkyl radicals contain from 1 to 3 separated and/or fused rings and from 3 to about 18 carbon atoms, preferably from 3 to 10 carbon atoms, such as cyclopropyl, cyclohexyl or adamantyl. In a particular embodiment, the cycloalkyl radical contains from 3 to about 6 carbon atoms.

“Aryl” refers to single and multiple ring radicals, including multiple ring radicals that contain separate and/or fused aryl groups. Typical aryl groups contain from 1 to 3 separated or fused rings and from 6 to about 18 carbon ring atoms, such as phenyl, naphthyl (e.g. 2-naphthyl), indenyl, fenanthryl or anthracyl radical.

“Heterocyclyl” includes both aromatic and non-aromatic heterocyclic groups.

“Aromatic Heterocyclyl” or “Heteroaryl” refers to heteroaromatic groups containing from 1 to 3 separated and/or fused rings and from 3 to about 18 ring atoms. Preferably heteroaromatic groups contain from 5 to about 10 ring atoms. Suitable heteroaromatic groups in the compounds of the present invention contain one, two or three heteroatoms selected from N, O or S atoms and include, e.g., coumarinyl including 8-coumarinyl, quinolyl including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothienyl, benzothiazolyl, benzoxazolyl, quinazolinyl, quinoxalinyl, naphthyridinyl, and furopyridinyl.

“Non-aromatic Heterocyclyl” refers to heteroalicyclic groups containing from 1 to 3 separated and/or fused rings and from 3 to about 18 ring atoms. Preferably heteroalicyclic groups contain from 5 to about 10 ring atoms. Suitable heteroalicyclic groups in the compounds of the present invention contain one, two or three heteroatoms selected from N, O or S atoms and include, e.g., pyrrolidinyl, tetrahydrofuryl, dihydrofuryl, tetrahydrothienyl, tetrahydrothiopyranyl, piperidyl, morpholinyl, thiomorpholinyl, thioxanyl, piperazinyl, azetidinyl, oxetanyl, thietanyl, homopiperidinyl, oxepanyl, thiepanyl, azepinyl, oxazepinyl, diazepinyl, thiazepinyl, 1,2,3,6-tetrahydropyridyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, pyrazolidinyl, imidazolinyl, imidazolidinyl, 3-azabicyclo[3.1.0]hexyl, 3-azabicyclo[4.1.0]heptyl, 3H-indolyl, and quinolizinyl.

“Alkoxy” refers to a radical of the formula —ORₐ where Rₐ is an alkyl radical as defined above having one or more (e.g., 1, 2, 3 or 4) oxygen linkages and typically from 1 to about 12, 1 to about 8 or 1 to about 6 carbon atoms, e.g., methoxy, ethoxy, propoxy, etc.

“Aryloxy” refers to a radical of formula O-aryl, where aryl is as previously defined. Some examples of aryloxy compounds are 0-phenyl (i.e. phenoxy), O-p-tolyl, —O-m-tolyl, —O-o-tolyl or O-naphthyl.

“Amino” refers to a radical of the formula —NH₂, —NHRₐ or —NRₐR_{b}, optionally quaternized. In an embodiment of the invention each of Rₐ and R_{b} is independently selected from hydrogen and an alkyl radical as defined above. Therefore, examples of amino groups are, methylamino, ethylamino, dimethylamino, diethylamino, propylamino, etc. . . . .

“Halogen”, “halo” or “hal” refers to bromo, chloro, iodo or fluoro.

“Fused ring system” refers to a polycyclic ring system that contains fused rings. Typically, the fused ring system contains 2 or 3 rings and/or up to 18 ring atoms. As defined above, cycloalkyl radicals, aryl radicals and heterocyclyl radicals may form fused ring systems. Thus, fused ring system may be aromatic, partially aromatic or not aromatic and may contain heteroatoms. A spiro ring system is not a fused-polycyclic by this definition, but fused polycyclic ring systems of the invention may themselves have spiro rings attached thereto via a single ring atom of the system. Examples of fused ring systems are, but are not limited to, adamantyl, naphthyl (e.g. 2-naphthyl), indenyl, fenanthryl, anthracyl, pyrenyl, benzimidazole, benzothiazole, etc.

Unless otherwise stated specifically in the specification, all the groups may be optionally substituted, if applicable. References herein to substituted groups in the compounds of the present invention refer to the specified moiety that may be substituted at one or more (e.g., 1, 2, 3 or 4) available positions by one or more suitable groups, e.g., halogen such as fluoro, chloro, bromo and iodo; cyano; hydroxyl; nitro; azido; acyl, such as alkanoyl, e.g. a C₁₋₆ alkanoyl group, and the like; carboxamido; alkyl groups including those groups having 1 to about 12 carbon atoms or from 1 to about 6 carbon atoms and more preferably 1-3 carbon atoms; alkenyl and alkynyl groups including groups having one or more (e.g., 1, 2, 3 or 4) unsaturated linkages and from 2 to about 12 carbon or from 2 to about 6 carbon atoms; alkoxy groups having one or more (e.g., 1, 2, 3 or 4) oxygen linkages and from 1 to about 12 carbon atoms or 1 to about 6 carbon atoms; aryloxy such as phenoxy; alkylthio groups including those moieties having one or more (e.g., 1, 2, 3 or 4) thioether linkages and from 1 to about 12 carbon atoms or from 1 to about 6 carbon atoms; alkylsulfinyl groups including those moieties having one or more (e.g., 1, 2, 3 or 4) sulfinyl linkages and from 1 to about 12 carbon atoms or from 1 to about 6 carbon atoms; alkylsulfonyl groups including those moieties having one or more (e.g., 1, 2, 3 or 4) sulfonyl linkages and from 1 to about 12 carbon atoms or from 1 to about 6 carbon atoms; aminoalkyl groups such as groups having one or more (e.g., 1, 2, 3 or 4) N atoms and from 1 to about 12 carbon atoms or from 1 to about 6 carbon atoms; carbocylic aryl having 6 or more carbons, particularly phenyl or naphthyl and aralkyl such as benzyl.

The term “salt” must be understood as any form of a compound used in accordance with this invention in which said compound is in ionic form or is charged and coupled to a counter-ion (a cation or anion) or is in solution. This definition also includes quaternary ammonium salts and complexes of the molecule with other molecules and ions, particularly, complexes formed via ionic interactions. The definition includes in particular physiologically acceptable salts; this term must be understood as equivalent to “pharmacologically acceptable salts” or “pharmaceutically acceptable salts”.

The term “pharmaceutically acceptable salts” in the context of this invention means any salt that is tolerated physiologically (normally meaning that it is not toxic, particularly, as a result of the counter-ion) when used in an appropriate manner for a treatment, applied or used, particularly, in humans and/or mammals. These physiologically acceptable salts may be formed with cations or bases and, in the context of this invention, are understood to be salts formed by at least one compound used in accordance with the invention normally an acid (deprotonated) such as an anion and at least one physiologically tolerated cation, preferably inorganic, particularly when used on humans and/or mammals. Salts with alkali and alkali earth metals are preferred particularly, as well as those formed with ammonium cations (NH₄⁺). Preferred salts are those formed with (mono) or (di)sodium, (mono) or (di)potassium, magnesium or calcium. These physiologically acceptable salts may also be formed with anions or acids and, in the context of this invention, are understood as being salts formed by at least one compound used in accordance with the invention normally protonated, for example in nitrogen such as a cation and at least one physiologically tolerated anion, particularly when used on humans and/or mammals. This definition specifically includes in the context of this invention a salt formed by a physiologically tolerated acid, i.e. salts of a specific active compound with physiologically tolerated organic or inorganic acids particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “solvate” in accordance with this invention should be understood as meaning any form of a compound in accordance with the invention in which said compound is bonded by a non-covalent bond to another molecule (normally a polar solvent), including especially hydrates and alcoholates, like for example, methanolate. A preferred solvate is the hydrate.

Any compound that is a prodrug of a compound referred to herein is also within the scope of the invention. The term “prodrug” is used in its broadest sense and encompasses those derivatives that are converted in vivo to the compounds of the invention. Examples of prodrugs include, but are not limited to, derivatives of the compounds referred to herein such as compounds of formula (I) that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described in “Burger's Medicinal Chemistry, Drug Discovery and Development” 7th ed. (Donald J. Abraham ed., 2010, Wiley), “Design of Prodrugs” (H. Bundgaard ed., 1985, Elsevier), “A Textbook of Drug Design and Development” (P. Krogsgaard-Larsen and H. Bundgaard eds., 1991, Harwood Academic Publishers; Chapter 5: “Design and Applications of Prodrugs”, p. 113-191) and “Textbook of Drug Design and Discovery” 4th ed. (P. Krogsgaard-Larsen et al. ed., 2010, Taylor & Francis).

Any compound referred to herein is intended to represent such specific compound as well as certain variations or forms. In particular, compounds referred to herein may have asymmetric centres and therefore exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same as, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Furthermore, any compound referred to herein may exist as tautomers. Specifically, the term tautomer refers to one of two or more structural isomers of a compound that exist in equilibrium and are readily converted from one isomeric form to another. Common tautomeric pairs are enamine-imine, amide-imidic acid, keto-enol, lactam-lactim, etc.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of the invention or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrug.

As used herein, the terms “treat”, “treating” and “treatment” include the eradication, removal, reversion, alleviation, modification, or control of pain after its onset.

As used herein, the terms “prevention”, “preventing”, “preventive” “prevent” and “prophylaxis” refer to the capacity of a therapeutic to avoid, minimize or difficult the onset or development of a disease or condition before its onset, in this case pain.

Therefore, by “treating” or “treatment” and/or “preventing” or “prevention”, as a whole, is meant at least a suppression or an amelioration of the symptoms associated with the condition afflicting the subject, where suppression and amelioration are used in a broad sense to refer to at least a reduction in the magnitude of a parameter, e.g., symptom associated with the condition being treated, such as pain. As such, the method of the present invention also includes situations where the condition is completely inhibited, e.g., prevented from happening, or stopped, e.g., terminated, such that the subject no longer experiences the condition. As such, the present method includes both preventing and managing pain, particularly, peripheral neuropathic pain, central neuropathic pain, allodynia, causalgia, hyperalgesia, hyperesthesia, hyperpathia, neuralgia, neuritis or neuropathy.

As used herein, the term “potentiating the analgesic effect of a Gabapentinoid” refers to the increase in the effectiveness of the analgesic effect of said Gabapentinoid produced by sigma ligands. In an embodiment of the invention, said potentiating effect induces an increase in the analgesic effect of the Gabapentinoid by a factor of 1.2, 1.5, 2, 3, 4 or more when compared with the Gabapentinoid when administered in isolation. The measurement can be done following any known method in the art. As used herein, the term “potentiating the analgesic effect of a Sigma ligand” refers to the increase in the effectiveness of the analgesic effect of said Sigma ligand produced by Gabapentinoid. In an embodiment of the invention said potentiating effect induces an increase in the analgesic effect of the Sigma ligand by a factor of 1.2, 1.5, 2, 3, 4 or more when compared with the Sigma ligand when administered in isolation. The measurement can be done following any known method in the art.

As above mentioned, the Sigma ligands of general formula (I) surprisingly potentiate the analgesic effect of Gabapentinoids, thus reducing the doses needed to obtain effective analgesia of the latter. In preferred variants, the synergistic combination of the invention comprises at least one Gabapentinoid and at least one Sigma ligand of general formula (I), said Gabapentinoid being present in the combination in a subactive dose or in a non-effective amount (that is, in a dose or amount that is not active or effective to provide the desired effect when used alone).

“Synergy” may be defined as the interaction of multiple elements in a system to produce an effect different from or greater than the sum of their individual effects. Thus, the combinations of the present invention are synergistic.

In a preferred embodiment, R₁ in the compounds of general formula (I) is selected from H, —COR₈, and substituted or unsubstituted alkyl. More preferably, R₁ is selected from H, methyl and acetyl. A more preferred embodiment is when R₁ is H.

In another preferred embodiment, R₂ in the compounds of formula (I) represents H or substituted or unsubstituted alkyl, more preferably methyl.

In a particular embodiment of the invention, R₃ and R₄ in the compounds of formula (I) are situated in the meta and para positions of the phenyl group, and preferably, they are selected independently from halogen and substituted or unsubstituted alkyl.

In an especially preferred embodiment of the invention, in the compounds of formula (I) both R₃ and R₄ together with the phenyl group form an optionally substituted fused ring system. More preferably, said fused ring system is selected from a substituted or unsubstituted fused aryl group and a substituted or unsubstituted aromatic or partially aromatic fused heterocyclyl group. Said fused ring system preferably contains two rings and/or from 9 to about 18 ring atoms, more preferably 9 or 10 ring atoms. Even more preferably, the fused ring system is naphthyl, especially a 2-naphthyl ring system, substituted or unsubstituted.

Also in the compounds of formula (I), embodiments where n is selected from 2, 3 or 4 are preferred in the context of the present invention, more preferably n is 2.

In another embodiment it is preferred in the compounds of formula (I) that R₅ and R₆ are, each independently, C₁₋₈alkyl, or together with the nitrogen atom to which they are attached form a substituted or unsubstituted heterocyclyl group, in particular a group chosen among morpholinyl, piperidinyl, and pyrrolidinyl group. More preferably, R₅ and R₆ together form a morpholine-4-yl group.

In additional preferred embodiments, the preferences described above for the different substituents are combined. The present invention is also directed to such combinations of preferred substitutions in the formula (I) above.

In preferred variants of the invention, the Sigma ligand of general formula (I) is selected from:
[1] 4-{2-(1-(3,4-dichlorophenyl)-5-methyl-1H pyrazol-3-yloxy)ethyl}morpholine,
[2] 2-[1-(3,4-Dichlorophenyl)-5-methyl-1H-pyrazol-3-yloxy]-N,N-diethylethanamine,
[3] 1-(3,4-Dichlorophenyl)-5-methyl-3-[2-(pyrrolidin-1-yl)ethoxy]-1H-pyrazole,
[4] 1-(3,4-Dichlorophenyl)-5-methyl-3-[3-(pyrrolidin-1-yl)propoxy]-1H-pyrazole,
[5] 1-{2-[1-(3,4-Dichlorophenyl)-5-methyl-1H-pyrazol-3-yloxy]ethyl}piperidine,
[6] 1-{2-[1-(3,4-dichlorophenyl)-5-methyl-1H-pyrazol-3-yloxy]ethyl}-1H-imidazole,
[7] 3-{1-[2-(1-(3,4-Dichlorophenyl)-5-methyl-1H-pyrazol-3-yloxy)ethyl]piperidin-4-yl}-3H-imidazo[4,5-b]pyridine,
[8] 1-{2-[1-(3,4-Dichlorophenyl)-5-methyl-1H-pyrazol-3-yloxy]ethyl}-4-methylpiperazine,
[9] Ethyl 4-{2-[1-(3,4-dichlorophenyl)-5-methyl-1H-pyrazol-3-yloxy]ethyl}piperazine carboxylate,
[10] 1-(4-(2-(1-(3,4-dichlorophenyl)-5-methyl-1H-pyrazol-3-yloxy)ethyl)piperazin-1-yl)ethanone,
[11] 4-{2-[1-(4-Methoxyphenyl)-5-methyl-1H-pyrazol-3-yloxy]ethyl}morpholine,
[12] 1-(4-Methoxyphenyl)-5-methyl-3-[2-(pyrrolidin-1-yl)ethoxy]-1H-pyrazole,
[13] 1-(4-Methoxyphenyl)-5-methyl-3-[3-(pyrrolidin-1-yl)propoxy]-1H-pyrazole,
[14] 1-[2-(1-(4-Methoxyphenyl)-5-methyl-1H-pyrazol-3-yloxy)ethyl]piperidine,
[15] 1-{2-[1-(4-Methoxyphenyl)-5-methyl-1H-pyrazol-3-yloxy]ethyl}-1H-imidazole,
[16] 4-{2-[1-(3,4-Dichlorophenyl)-5-phenyl-1H-pyrazol-3-yloxy]ethyl}morpholine,
[17] 1-(3,4-Dichlorophenyl)-5-phenyl-3-[2-(pyrrolidin-1-yl)ethoxy]-1H-pyrazole,
[18] 1-(3,4-Dichlorophenyl)-5-phenyl-3-[3-(pyrrolidin-1-yl)propoxy]-1H-pyrazole,
[19] 1-{2-[1-(3,4-Dichlorophenyl)-5-phenyl-1H-pyrazol-3-yloxy]ethyl}piperidine,
[20] 1-{2-[1-(3,4-Dichlorophenyl)-5-phenyl-1H-pyrazol-3-yloxy]ethyl}-1H-imidazole,
[21] 2-{2-[1-(3,4-dichlorophenyl)-5-phenyl-1H-pyrazol-3-yloxy]ethyl}-1,2,3,4-tetrahydroisoquinoline,
[22] 4-{4-[1-(3,4-Dichlorophenyl)-5-methyl-1H-pyrazol-3-yloxy]butyl}morpholine,
[23] 1-(3,4-Dichlorophenyl)-5-methyl-3-[4-(pyrrolidin-1-yl)butoxy]-1H-pyrazole,
[24] 1-{4-[1-(3,4-Dichlorophenyl)-5-methyl-1H-pyrazol-3-yloxy]butyl}piperidine,
[25]1-{4-[1-(3,4-Dichlorophenyl)-5-methyl-1H-pyrazol-3-yloxy]butyl}-4-methylpiperazine,
[26] 1-{4-[1-(3,4-Dichlorophenyl)-5-methyl-1H-pyrazol-3-yloxy]butyl}-1H-imidazole,
[27] 4-[1-(3,4-Dichlorophenyl)-5-methyl-1H-pyrazol-3-yloxy]-N,N-diethylbutan-1-amine,
[28]1-{4-[1-(3,4-dichlorophenyl)-5-methyl-1H-pyrazol-3-yloxy]butyl}-4-phenylpiperidine,
[29] 1-{4-[1-(3,4-dichlorophenyl)-5-methyl-1H-pyrazol-3-yloxy]butyl}-6,7-dihydro-1H-indol-4(5H)-one,
[30] 2-{4-[1-(3,4-dichlorophenyl)-5-methyl-1H-pyrazol-3-yloxy]butyl}-1,2,3,4-tetrahydroisoquinoline,
[31] 4-{2-[1-(3,4-dichlorophenyl)-5-isopropyl-1H-pyrazol-3-yloxy]ethyl}morpholine,
[32] 2-[1-(3,4-Dichlorophenyl)-5-isopropyl-1H-pyrazol-3-yloxy]-N,N-diethylethanamine,
[33] 1-(3,4-Dichlorophenyl)-5-isopropyl-3-[2-(pyrrolidin-1-yl)ethoxy]-1H-pyrazole,
[34] 1-(3,4-Dichlorophenyl)-5-isopropyl-3-[3-(pyrrolidin-1-yl)propoxy]-1H-pyrazole,
[35] 1-{2-[1-(3,4-Dichlorophenyl)-5-isopropyl-1H-pyrazol-3-yloxy]ethyl}piperidine,
[36] 2-{2-[1-(3,4-dichlorophenyl)-5-isopropyl-1H-pyrazol-3-yloxy]ethyl}-1,2,3,4-tetrahydroisoquinoline,
[37] 4-{2-[1-(3,4-dichlorophenyl)-1H-pyrazol-3-yloxy]ethyl}morpholine,
[38] 2-[1-(3,4-dichlorophenyl)-1H-pyrazol-3-yloxy] N,N-diethylethanamine,
[39] 1-(3,4-dichlorophenyl)-3-[2-(pyrrolidin-1-yl)ethoxy]-1H-pyrazole,
[40] 1-{2-[1-(3,4-dichlorophenyl)-1H-pyrazol-3-yloxy]ethyl}piperidine,
[41] 1-(3,4-dichlorophenyl)-3-[3-(pyrrolidin-1-yl)propoxy]-1H-pyrazole,
[42]1-{2-[1-(3,4-Dichlorophenyl)-5-methyl-1H-pyrazol-3-yloxy]ethyl}piperazine,
[43] 1-{2-[1-(3,4-Dichlorophenyl)-5-methyl-1H-pyrazol-3-yloxy]ethyl}pyrrolidin-3-amine,
[44] 4-{2-[1-(3,4-Dichlorophenyl)-4,5-dimethyl-1H-pyrazol-3-yloxy]ethyl}morpholine,
[46] 2-[1-(3,4-Dichlorophenyl)-4,5-dimethyl-1H-pyrazol-3-yloxy]-N,N-diethylethanamine,
[47] 1-(3,4-Dichlorophenyl)-4,5-dimethyl-3-[2-(pyrrolidin-1-Methoxy]-1H-pyrazole,
[48] 1-(3,4-Dichlorophenyl)-4,5-dimethyl-3-[3-(pyrrolidin-1-yl)propoxy]-1H-pyrazole,
[49] 1-{2-[1-(3,4-Dichlorophenyl)-4,5-dimethyl-1H-pyrazol-3-yloxy]ethyl}piperidine,
[50] 4-{4-[1-(3,4-dichlorophenyl)-1H-pyrazol-3-yloxy]butyl}morpholine,
[51](2S,6R)-4-{4-[1-(3,4-dichlorophenyl)-1H-pyrazol-3-yloxy]butyl}-2,6-dimethylmorpholine,
[52] 1-{4-[1-(3,4-Dichlorophenyl)-1H-pyrazol-3-yloxy]butyl}piperidine,
[53] 1-(3,4-Dichlorophenyl)-3-[4-(pyrrolidin-1-yl)butoxy]-1H-pyrazole,
[55] 4-[1-(3,4-dichlorophenyl)-1H-pyrazol-3-yloxy]-N,N-diethylbutan-1-amine,
[56] N-benzyl-4-[1-(3,4-dichlorophenyl)-1H-pyrazol-3-yloxy]-N-methylbutan-1-amine,
[57]4-[1-(3,4-dichlorophenyl)-1H-pyrazol-3-yloxy]-N-(2-methoxyethyl)-N-methylbutan-1-amine,
[58] 4-{4-[1-(3,4-dichlorophenyl)-1H-pyrazol-3-yloxy]butyl}thiomorpholine,
[59]1-[1-(3,4-Dichlorophenyl)-5-methyl-3-(2-morpholinoethoxy)-1H-pyrazol-4-yl]ethanone,
[60]1-{1-(3,4-dichlorophenyl)-5-methyl-3-[2-(pyrrolidin-1-Methoxy]-1H-pyrazol-4-yl}ethanone,
[61] 1-{1-(3,4-dichlorophenyl)-5-methyl-3-[2-(piperidin-1-yl)ethoxy]-1H-pyrazol-4-yl}ethanone,
[62] 1-{1-(3,4-dichlorophenyl)-3-[2-(diethylamino)ethoxy]-5-methyl-1H-pyrazol-4-yl}ethanone,
[63] 4-{2-[5-Methyl-1-(naphthalen-2-yl)-1H-pyrazol-3-yloxy]ethyl}morpholine,
[64] N,N-Diethyl-2-[5-methyl-1-(naphthalen-2-yl)-1H-pyrazol-3-yloxy] ethanamine,
[65] 1-{2-[5-Methyl-1-(naphthalen-2-yl)-1H-pyrazol-3-yloxy]ethyl}piperidine, and
[66] 5-Methyl-1-(naphthalen-2-yl)-3-[2-(pyrrolidin-1-yl)ethoxy]-1H-pyrazole,

or a pharmaceutically acceptable salt, isomer, solvate or prodrug thereof.

In a preferred variant of the invention, the Sigma ligand of general formula (I) is 4-{2-[5-Methyl-1-(naphthalen-2-yl)-1H-pyrazol-3-yloxy]ethyl}morpholine or a salt thereof.

Preferably, the compound of general formula (I) used is 4-{2-[5-Methyl-1-(naphthalen-2-yl)-1H-pyrazol-3-yloxy]ethyl}morpholine hydrochloride.

These particular compounds are designated in the examples of the present invention as compound 63 and compound 63.HCl.

The compounds of general formula (I) and their salts or solvates can be prepared as disclosed in the previous application WO2006/021462.

By “Gabapentinoid” is meant any member of the class of compounds that (i) are α2δ subunit calcium channel ligands, (ii) have an inhibition constant (Ki) of 1000 nM or less, desirably less than 100 nM. The term “Gabapentinoids” also refer to compounds binding at the same site as Gabapentin, or, alternatively, acting like Gabapentin or Pregabalin.

In one embodiment, Gabapentinoids are compounds that are derived from or based on gamma-aminobutyric acid (GABA), also known as GABA analogues. These compounds are either readily available or can be readily synthesized using known methods. Preferred gabapentin analogues as well as their respective syntheses are described in J. S. Bryans et al., Biorg. Med. Chem. Lett. 1999, 9, 2329-2334; J. S. Bryans, D. J. Wustrow, Med. Res. Rev. 1999, 19, 149-177; J. S. Bryans et al., J. Org. Chem. 1998, 41, 1838-1845; U.S. Pat. No. 4,024,175, WO 92/09560, WO 93/23383, WO 99/61424, WO 99/31057, WO 99/31074, WO 99/31075, WO 99/21824, WO 00/73259, WO 00/73300, WO 00/73296, WO 00/31020, U.S. Pat. No. 6,166,072 and WO 02/085839.

The term “analogues” as used in this application is defined here as meaning a chemical compound that is a derivative of a compound which has similar biochemical activity with respect to that compound. The term “derivative” preferably includes entities structurally derived from a given compound i.e. a chemical compound having undergone a chemical derivatization such as substitution or addition of a further chemical group to change (for pharmaceutical use) any of its physico-chemical properties, such as solubility or bioavailability. Derivatives include so-called prodrugs.

In a particular embodiment of the present invention, the Gabapentinoid is selected from the group consisting of Gabapentin, Pregabalin, Atagabalin, Imagabalin, DS-5565 (from Daiichi Sankyo) and Gabapentin enacarbil or a pharmaceutically acceptable salt, isomer, prodrug or solvate thereof.

In a preferred embodiment of the present invention, the Gabapentinoid is selected from the group consisting of Gabapentin and Pregabalin or a pharmaceutically acceptable salt, isomer, prodrug or solvate thereof.

A preferred embodiment refers to the synergistic combination of the invention comprising 4-{2-[5-methyl-1-(naphthalen-2-yl)-1H-pyrazol-3-yloxy]ethyl}morpholine or a pharmaceutically acceptable salt, isomer, prodrug or solvate thereof and Gabapentin or a pharmaceutically acceptable salt, isomer, prodrug or solvate thereof.

A more preferred embodiment refers to the synergistic combination of the invention comprising 4-{2-[5-methyl-1-(naphthalen-2-yl)-1H-pyrazol-3-yloxy]ethyl}morpholine hydrochloride and Gabapentin.

A preferred embodiment refers to the synergistic combination of the invention comprising 4-{2-[5-methyl-1-(naphthalen-2-yl)-1H-pyrazol-3-yloxy]ethyl}morpholine or a pharmaceutically acceptable salt, isomer, prodrug or solvate thereof and Pregabalin or a pharmaceutically acceptable salt, isomer, prodrug or solvate thereof.

A more preferred embodiment refers to the synergistic combination of the invention comprising 4-{2-[5-methyl-1-(naphthalen-2-yl)-1H-pyrazol-3-yloxy]ethyl}morpholine hydrochloride and Pregabalin.

The present invention refers also to medicaments or pharmaceutical compositions comprising at least one Sigma ligand of general formula (I) as defined above, or a pharmaceutically acceptable salt, isomer, prodrug or solvate thereof, and at least one Gabapentinoid combined jointly or separately, together with at least a pharmaceutically acceptable excipient.

The term “excipient” refers to components of a drug compound other than the active ingredient (definition obtained from the European Medicines Agency—EMA). They preferably include a “carrier, adjuvant and/or vehicle”. Carriers are forms to which substances are incorporated to improve the delivery and the effectiveness of drugs. Drug carriers are used in drug-delivery systems such as the controlled-release technology to prolong in vivo drug actions, decrease drug metabolism, and reduce drug toxicity. Carriers are also used in designs to increase the effectiveness of drug delivery to the target sites of pharmacological actions (U.S. National Library of Medicine. National Institutes of Health). Adjuvant is a substance added to a drug product formulation that affects the action of the active ingredient in a predictable way. Vehicle is an excipient or a substance, preferably without therapeutic action, used as a medium to give bulk for the administration of medicines (Stedman's Medical Spellchecker, 2006 Lippincott Williams & Wilkins). Such pharmaceutical carriers, adjuvants or vehicles can be sterile liquids, such as water and oils, including those of petroleum, animal, vegetable or synthetic origin, such as peanut oil, soybean oil, mineral oil, sesame oil and the like, excipients, disgregants, wetting agents or diluents. Suitable pharmaceutical carriers are described in “Remington's Pharmaceutical Sciences” by E. W. Martin. The selection of these excipients and the amounts to be used will depend on the form of application of the pharmaceutical composition.

The pharmaceutical composition according to the present invention can be adapted to any form of administration, be it orally or parenterally, for example pulmonarily, nasally, rectally and/or intravenously. Therefore, the formulation according to the present invention may be adapted for topical or systemic application, particularly for dermal, subcutaneous, intramuscular, intra-articular, intraperitoneal, pulmonary, buccal, sublingual, nasal, percutaneous, vaginal, oral or parenteral application. The preferred form of rectal application is by means of suppositories.

Suitable preparations for oral applications are tablets, pills, chewing gums, capsules, granules, drops or syrups. Suitable preparations for parenteral applications are solutions, suspensions, reconstitutable dry preparations or sprays.

The synergistic combination of the invention may be formulated as deposits in dissolved form or in patches, for percutaneous application. Skin applications include ointments, gels, creams, lotions, suspensions or emulsions.

The synergistic combination of the invention may be formulated for its simultaneous, separate or sequential administration, with at least a pharmaceutically acceptable excipient. This has the implication that the synergistic combination of the Sigma ligand of general formula (I) and the Gabapentinoid may be administered:
a) As a combination that is being part of the same medicament formulation, both being then administered always simultaneously.
b) As a combination of two units, each with one of them giving rise to the possibility of simultaneous, sequential or separate administration. In a particular embodiment, the Sigma ligand of general formula (I) is independently administered from the Gabapentinoid (i.e in two units) but at the same time. In another particular embodiment, the sigma ligand of general formula (I) is administered first, and then the Gabapentinoid is separately or sequentially administered. In yet another particular embodiment, the Gabapentinoid is administered first, and then the Sigma ligand of general formula (I) is administered, separately or sequentially, as defined.

In a particular embodiment of the present invention, the pain is selected from peripheral and central neuropathic pain, allodynia, causalgia, hyperalgesia, hyperesthesia, hyperpathia, neuralgia, neuritis or neuropathy. More preferably, the pain is peripheral neuropathic pain, hyperalgesia or allodynia.

“Neuropathic pain” is defined by the IASP as “pain initiated or caused by a primary lesion or dysfunction in the nervous system” (IASP, Classification of chronic pain, 2^{nd} Edition, IASP Press (1994), 210). For the purpose of this invention this term is to be treated as synonymous to “Neurogenic Pain” which is defined by the IASP as “pain initiated or caused by a primary lesion, dysfunction or transitory perturbation in the peripheral or central nervous system”.

According to the IASP “peripheral neuropathic pain” is defined as “a pain initiated or caused by a primary lesion or dysfunction in the peripheral nervous system” and “peripheral neurogenic pain” is defined as “a pain initiated or caused by a primary lesion, dysfunction or transitory perturbation in the peripheral nervous system” (IASP, Classification of chronic pain, 2^{nd} Edition, IASP Press (1994), 213).

According to the IASP “allodynia” is defined as “a pain due to a stimulus which does not normally provoke pain” (IASP, Classification of chronic pain, 2^{nd} Edition, IASP Press (1994), 210).

According to the IASP “causalgia” is defined as “a syndrome of sustained burning pain, allodynia and hyperpathia after a traumatic nerve lesion, often combined with vasomotor and sudomotor dysfunction and later trophic changes” (IASP, Classification of chronic pain, 2^{nd} Edition, IASP Press (1994), 210).

According to the IASP “hyperalgesia” is defined as “an increased response to a stimulus which is normally painful” (IASP, Classification of chronic pain, 2^{nd} Edition, IASP Press (1994), 211).

According to the IASP “hyperesthesia” is defined as “increased sensitivity to stimulation, excluding the senses” (IASP, Classification of chronic pain, 2^{nd} Edition, IASP Press (1994), 211).

According to the IASP “hyperpathia” is defined as “a painful syndrome characterized by an abnormally painful reaction to a stimulus, especially a repetitive stimulus, as well as an increased threshold” (IASP, Classification of chronic pain, 2^{nd} Edition, IASP Press (1994), 212).

The IASP draws the following difference between “allodynia”, “hyperalgesia” and “hyperpathia” (IASP, Classification of chronic pain, 2^{nd} Edition, IASP Press (1994), 212):

| | | |
|---|---|---|
| Allodynia | Lowered threshold | Stimulus and response |
| | | mode differ |
| Hyperalgesia | Increased response | Stimulus and response rate |
| | | are the same |
| Hyperpathia | Raised threshold | Stimulus and response rate |
| | Increased response | may be the same or |
| | | different |

According to the IASP “neuralgia” is defined as “pain in the distribution of a nerve or nerves” (IASP, Classification of chronic pain, 2^{nd} Edition, IASP Press (1994), 212).

According to the IASP “neuritis” is defined as “inflammation of a nerve or nerves” (IASP, Classification of chronic pain, 2^{nd} Edition, IASP Press (1994), 212).

According to the IASP “neuropathy/neuritis” is defined as “a disturbance of function or pathological change in a nerve: in one nerve mononeuropathy, in several nerves mononeuropthy multiplex, if diffuse and bilateral, polyneuropathy” (IASP, Classification of chronic pain, 2^{nd} Edition, IASP Press (1994), 212).

Another aspect of the invention is a method of treatment and/or prophylaxis of a patient suffering from pain, or likely to suffer pain, the method comprising administering to the patient in need of such a treatment or prophylaxis a therapeutically effective amount of a synergistic combination comprising at least one Sigma ligand of general formula (I) as defined above, or a pharmaceutically acceptable salt, isomer, prodrug or solvate thereof, and at least one Gabapentinoid.

By an “effective” amount or a “therapeutically effective amount” of a drug or pharmacologically active agent is meant a nontoxic but sufficient amount of the drug or agent to provide the desired effect. In the combination therapy of the present invention, an “effective amount” of one component of the combination (i.e. Sigma ligand of general formula (I) or Gabapentinoid) is the amount of that compound that is effective to provide the desired effect when used in combination with the other component of the combination (i.e. Gabapentinoid or Sigma ligand of general formula (I)). The amount that is “effective” will vary from subject to subject, depending on the age and general condition of the individual, the particular active agent or agents, and the like. Thus, it is not always possible to specify an exact “effective amount”. However, an appropriate “effective” amount in any individual case may be determined by one of ordinary skill in the art using routine experimentation.

According to the present invention the dosage of the Gabapentinoid can be reduced when combined with a Sigma ligand of general formula (I), and therefore attaining the same analgesic effect with a reduced dosage, and thus attenuating the adverse effects.

For example, the dosage regime that must be administered to the patient will depend on the patient's weight, the type of application, the condition and severity of the disease. A preferred dosage regime comprises an administration of a Sigma compound of general formula (I) within a range of 0.5 to 100 mg/kg and of the Gabapentinoid from 0.15 to 100 mg/kg. The administration may be performed once or in several occasions.

Having described the present invention in general terms, it will be more easily understood by reference to the following examples which are presented as an illustration and are not intended to limit the present invention.

### EXAMPLES

### Example 1

### Synthesis of 4-{2-[5-Methyl-1-(naphthalen-2-yl)-1H-pyrazol-3-yloxy]ethyl}morpholine (compound 63) and its hydrochloride salt

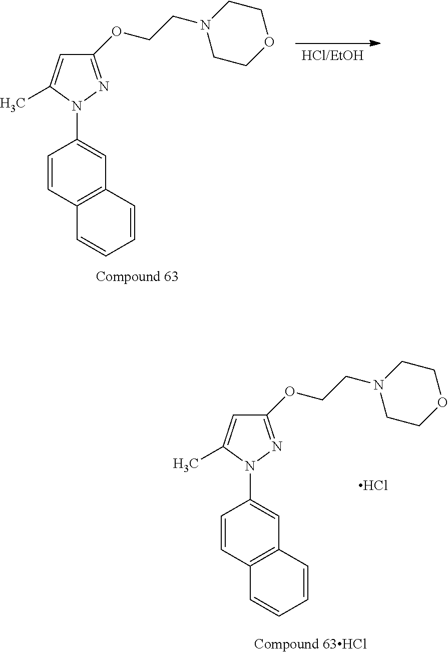 [SMILES: Cc1cc(OCCN2CCOCC2)nn1-c1ccc2ccccc2c1 ; Cc1cc(OCCN2CCOCC2)nn1-c1ccc2ccccc2c1 ; Cl]

Compound 63 can be prepared as disclosed in the previous application WO2006/021462. Its hydrochloride can be obtained according the following procedure:

Compound 63 (6.39 g) was dissolved in ethanol saturated with HCl, the mixture was stirred then for some minutes and evaporated to dryness. The residue was crystallized from isopropanol. The mother liquors from the first crystallization afforded a second crystallization by concentrating. Both crystallizations taken together yielded 5.24 g (63%) of the corresponding hydrochloride salt (m.p.=197-199° C.)

¹H-NMR (DMSO-d₆) δ ppm: 10.85 (bs, 1H), 7.95 (m, 4H), 7.7 (dd, J=2, 2, 8.8 Hz, 1H), 7.55 (m, 2H), 5.9 (s, 1H), 4.55 (m, 2H), 3.95 (m, 2H), 3.75 (m, 2H), 3.55-3.4 (m, 4H), 3.2 (m, 2H), 2.35 (s, 3H).

HPLC purity: 99.8%

### Example 2

### Assessment of Analgesia in the Treatment Post-Operative Pain

### 2.1 General Protocol

The induction of anesthesia in rats was performed with 3% isofluran for veterinary use, employing an Ohmeda vaporizer and an anesthesia chamber. Anesthesia was kept during the surgical operation by a tube which directs the isofluran vapors to the animal's snout. Once the rats were anesthetized, they were laid down in a prone position and their right hind paws were cleaned out with alcohol.

Then, a skin incision in the hindpaw of about 10 mm was made by means of a scalpel, starting about 5 mm from the heel and extending toward the toes. Fascia was located and by means of curve scissors muscle was elevated and a longitudinal incision of about 5 mm was made, thus the muscle origin and insertion remained intact. The skin of the paw was stitched with a suturing stitch with breaded silk (3.0) and the wound was cleaned out with povidone.

The assessment was performed 30 minutes after the administration of product and always 4 hours after the plantar incision. The analysis was carried out evaluating the mechanical allodynia. It was tested using von Frey filaments: Animals were placed in methacrylate cylinders on an elevated surface, with metallic mesh floor perforated in order to apply the filaments. After an acclimation period of about 30 minutes within the cylinders, both hindpaws were stimulated (the injured and the non-injured paw, serving the latter as control), starting with the lowest force filament (0.4 g) and reaching a 15 g filament. The animal's response to pain was manifested by the withdrawal of the paw as a consequence of the painful stimulus caused by a filament.

### 2.2 Combination of Compound 63.HCl and Preciabalin

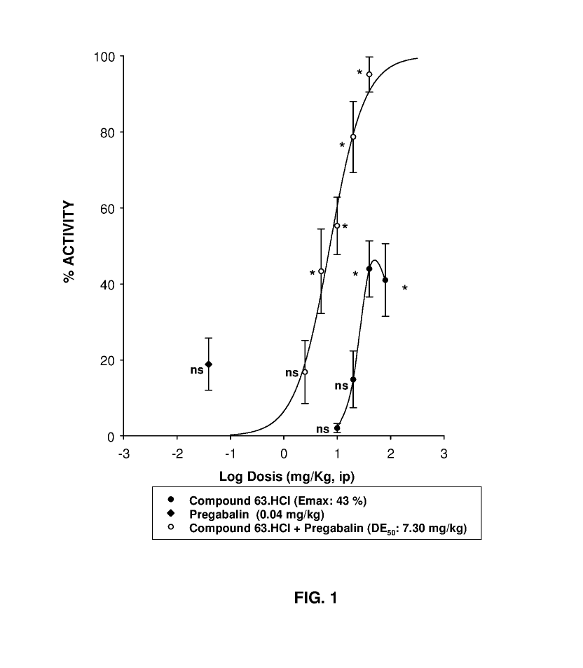

The efficacy of the combined use of Pregabalin and compound 63.HCl was tested at different doses of compound 63.HCl (5, 10, 20, 40 and 80 mg/kg), while the Pregabalin dose remained constant (0.04 mg/kg). The administrations were performed 3.5 hours after surgery. The treated subjects were tested according to the mechanical allodynia protocol above (FIG. 1).

### 2.3 Combination of Compound 63.HCl and Gabapentin

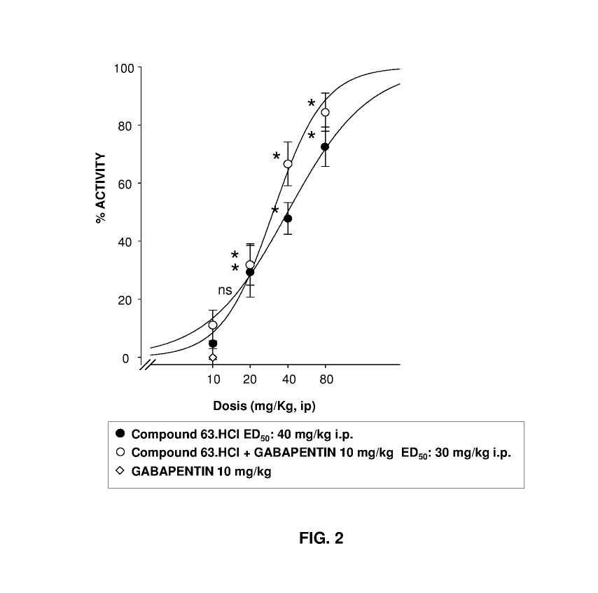

The efficacy of the combined use of Gabapentin and compound 63.HCl was tested at different doses of compound 63.HCl (10, 20, 40 and 80 mg/kg), while the Gabapentin dose remained constant (10 mg/kg). The administrations were performed 3.5 hours after surgery. The treated subjects were tested according to the mechanical allodynia protocol above (FIG. 2).

### Conclusions:

As shown in FIG. 1, compound 63.HCl produced a dose dependent effect with a maximum effect of 43%. The Figure also shows Pregabalin, in a sub-active dose (0.04 mg/kg) which produced a non-significant effect. Finally it can be seen that the combination Pregabalin (in a sub-active dose) and compound 63.HCl produced a dose-dependent effect with ED50=7.3 mg/kg. Therefore, compound 63.HCl and Pregabalin act synergically to produce analgesia in the treatment of post-operative pain.

As shown in FIG. 2, compound 63.HCl produced a dose dependent effect with an ED50 of 40 mg/kg. Gabapentin produced a non-significant effect at the dose of 10 mg/kg (0±2%). The combination gabapentin+compound 63.HCl produced a dose-dependent effect with higher potency than the compound 63.HCl alone (ED50=30 mg/kg).

### REFERENCES

Cobos, E. J., Entrena, J. M., Nieto, F. R., Cendan, C. M., Del Pozo, E. Pharmacology and therapeutic potential of Sigma(1) receptor ligands. Curr. Neuropharmacol. 2008; 6, 344-366.
Maurice, T., Su, T. P., The pharmacology of Sigma-1 receptors. Pharmacol. Ther. 2009; 124, 195-206.
Merskey et al.; “Part III: Pain Terms, A Current List with Definitions and Notes on Usage” (pp 209-214) Classification of Chronic Pain, Second Edition, IASP Task Force on Taxonomy, edited by H. Merskey and N. Bogduk, IASP Press, Seattle, ©1994.
Romero, L., Zamanillo, D., Nadal, X., Sanchez-Arroyos, R., Rivera-Arconada, I., Dordal, A., Montero, A., Muro, A., Bura, A., Segales, C., Laloya, M., Hernandez, E., Portillo-Salido, E., Escriche, M., Codony, X., Encina, G., Burgueno, J., Merlos, M., Baeyens, J., Giraldo, J., Lopez-Garcia, J., Maldonado, R., Plata-Salaman, C., Vela, J. Pharmacological properties of S1RA, a new Sigma-1 receptor antagonist that inhibits neuropathic pain and activity-induced spinal sensitization. Br. J. Pharmacol. 2012; doi: 10.1111/j.1476-5381.
Mark J. Field, Peter J. Cox, Emma Stott, Heather Melrose, James Offord, Ti-Zhi Su, Steve Bramwell, Laura Corradini, Steven England, Joanna Winks, Ross A. Kinloch, Jan Hendrich, Annette C. Dolphin, Tony Webb, and Dic Williams. Identification of the α2-δ-1 subunit of voltage dependent calcium channels as a molecular target for pain mediating the analgesic actions of pregabalin. PNAS; Nov. 14, 2006; vol. 103 no. 46; 17537-17542
Anthony Davies, Jan Hendrich, Alexandra Tran Van Minh, Jack Wratten, Leon Douglas and Annette C. Dolphin. Functional biology of the α₂δ subunits of voltage-gated calcium channels; TRENDS in Pharmacological Sciences; 2007; Vol 28 n° 5; 220-228.
Charles P. Taylor. Mechanisms of analgesia by gabapentin and pregabalin Calcium channel α₂-δ [Caᵥα₂-δ] ligands. Pain 142 (2009) 13-16
Danielle Perret and Z. David Luo. Targeting Voltage-Gated Calcium Channels for Neuropathic Pain Management; Journal of the American Society for Experimental Neuro Therapeutics; Vol. 6, 679-692, October 2009

1-16. (canceled)

17. A synergistic combination comprising at least one Gabapentinoid selected from a α2δ subunit calcium channel ligand having an inhibition constant (Ki) of 1000 nM or less and at least one Sigma ligand of general formula (I) 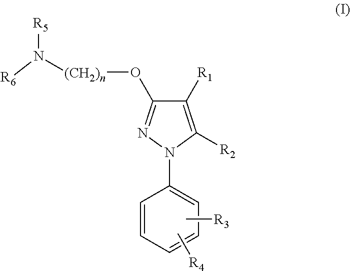
wherein,
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —ON, —OR₈, —OC(O)R₈, —S(O)ₜ—R₈, —NR₈R₉, —NR₈C(O)R₉, —NO₂, —N═CR₈R₉, and halogen;
R₂ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —ON, —OR_{B}, —OC(O)R₈, —S(O)ₜ—R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen, or together with the phenyl they form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclyalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₅R₉, and halogen;
or together form, with the nitrogen atom to which they are attached, a substituted or unsubstituted, aromatic or non-aromatic heterocyclyl group;
n is selected from 1, 2, 3, 4, 5, 6, 7 and 8;
t is 0, 1 or 2;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen,
or a pharmaceutically acceptable salt, isomer, prodrug or solvate thereof.

18. The synergistic combination according to claim 17, wherein R₁ is selected from H, —COR₈, and substituted or unsubstituted alkyl.

19. The synergistic combination according to claim 17, wherein R₂ is H or substituted or unsubstituted alkyl.

20. The synergistic combination according to claim 17, wherein R₃ and R₄ together with the phenyl group form a naphthyl ring system.

21. The synergistic combination according to claim 17, wherein n is selected from 2, 3, and 4.

22. The synergistic combination according to claim 17, wherein R₅ and R₆ together form a morpholine-4-yl group.

23. The synergistic combination according to claim 17, wherein the sigma ligand of general formula (I) is selected from:
4-{2-(1-(3,4-dichlorophenyl)-5-methyl-1H pyrazol-3-yloxy)ethyl}morpholine,
2-[1-(3,4-Dichlorophenyl)-5-methyl-1H-pyrazol-3-yloxy]-N,N-diethylethanamine,
1-(3,4-Dichlorophenyl)-5-methyl-3-[2-(pyrrolidin-1-yl)ethoxy]-1H-pyrazole,
1-(3,4-Dichlorophenyl)-5-methyl-3-[3-(pyrrolidin-1-yl)propoxy]-1H-pyrazole,
1-{2-[1-(3,4-Dichlorophenyl)-5-methyl-1H-pyrazol-3-yloxy]ethyl}piperidine,
1-{2-[1-(3,4-dichlorophenyl)-5-methyl-1H-pyrazol-3-yloxy]ethyl}-1H-imidazole,
3-{1-[2-(1-(3,4-Dichlorophenyl)-5-methyl-1H-pyrazol-3-yloxy)ethyl]piperidin-4-yl}-3H-imidazo[4,5-b]pyridine,
1-{2-[1-(3,4-Dichlorophenyl)-5-methyl-1H-pyrazol-3-yloxy]ethyl}-4-methylpiperazine,
Ethyl 4-{2-[1-(3,4-dichlorophenyl)-5-methyl-1H-pyrazol-3-yloxy]ethyl}piperazine carboxylate,
1-(4-(2-(1-(3,4-dichlorophenyl)-5-methyl-1H-pyrazol-3-yloxy)ethyl)piperazin-1-yl)ethanone,
4-{2-[1-(4-Methoxyphenyl)-5-methyl-1H-pyrazol-3-yloxy]ethyl}morpholine,
1-(4-Methoxyphenyl)-5-methyl-3-[2-(pyrrolidin-1-yl)ethoxy]-1H-pyrazole,
1-(4-Methoxyphenyl)-5-methyl-3-[3-(pyrrolidin-1-yl)propoxy]-1H-pyrazole,
1-[2-(1-(4-Methoxyphenyl)-5-methyl-1H-pyrazol-3-yloxy)ethyl]piperidine,
1-{2-[1-(4-Methoxyphenyl)-5-methyl-1H-pyrazol-3-yloxy]ethyl}-1H-imidazole,
4-{2-[1-(3,4-Dichlorophenyl)-5-phenyl-1H-pyrazol-3-yloxy]ethyl}morpholine,
1-(3,4-Dichlorophenyl)-5-phenyl-3-[2-(pyrrolidin-1-yl)ethoxy]-1H-pyrazole,
1-(3,4-Dichlorophenyl)-5-phenyl-3-[3-(pyrrolidin-1-yl)propoxy]-1H-pyrazole,
1-{2-[1-(3,4-Dichlorophenyl)-5-phenyl-1H-pyrazol-3-yloxy]ethyl}piperidine,
1-{2-[1-(3,4-Dichlorophenyl)-5-phenyl-1H-pyrazol-3-yloxy]ethyl}-1H-imidazole,
2-{2-[1-(3,4-dichlorophenyl)-5-phenyl-1H-pyrazol-3-yloxy]ethyl}-1,2,3,4-tetrahydroisoquinoline,
4-{4-[1-(3,4-Dichlorophenyl)-5-methyl-1H-pyrazol-3-yloxy]butyl}morpholine,
1-(3,4-Dichlorophenyl)-5-methyl-3-[4-(pyrrolidin-1-yl)butoxy]-1H-pyrazole,
1-{4-[1-(3,4-Dichlorophenyl)-5-methyl-1H-pyrazol-3-yloxy]butyl}piperidine,
1-{4-[1-(3,4-Dichlorophenyl)-5-methyl-1H-pyrazol-3-yloxy]butyl}-4-methylpiperazine,
1-{4-[1-(3,4-Dichlorophenyl)-5-methyl-1H-pyrazol-3-yloxy]butyl}-1H-imidazole,
4-[1-(3,4-Dichlorophenyl)-5-methyl-1H-pyrazol-3-yloxy]-N,N-diethylbutan-1-amine,
1-{4-[1-(3,4-dichlorophenyl)-5-methyl-1H-pyrazol-3-yloxy]butyl}-4-phenylpiperidine,
1-{4-[1-(3,4-dichlorophenyl)-5-methyl-1H-pyrazol-3-yloxy]butyl}-6,7-dihydro-1H-indol-4(5H)-one,
2-{4-[1-(3,4-dichlorophenyl)-5-methyl-1H-pyrazol-3-yloxy]butyl}-1,2,3,4-tetrahydroisoquinoline,
4-{2-[1-(3,4-dichlorophenyl)-5-isopropyl-1H-pyrazol-3-yloxy]ethyl}morpholine,
2-[1-(3,4-Dichlorophenyl)-5-isopropyl-1H-pyrazol-3-yloxy]-N,N-diethylethanamine,
1-(3,4-Dichlorophenyl)-5-isopropyl-3-[2-(pyrrolidin-1-yl)ethoxy]-1H-pyrazole,
1-(3,4-Dichlorophenyl)-5-isopropyl-3-[3-(pyrrolidin-1-yl)propoxy]-1H-pyrazole,
1-{2-[1-(3,4-Dichlorophenyl)-5-isopropyl-1H-pyrazol-3-yloxy]ethyl}piperidine,
2-{2-[1-(3,4-dichlorophenyl)-5-isopropyl-1H-pyrazol-3-yloxy]ethyl}-1,2,3,4-tetrahydroisoquinoline,
4-{2-[1-(3,4-dichlorophenyl)-1H-pyrazol-3-yloxy]ethyl}morpholine,
2-[1-(3,4-dichlorophenyl)-1H-pyrazol-3-yloxy]N,N-diethylethanamine,
1-(3,4-dichlorophenyl)-3-[2-(pyrrolidin-1-yl)ethoxy]-1H-pyrazole,
1-{2-[1-(3,4-dichlorophenyl)-1H-pyrazol-3-yloxy]ethyl}piperidine,
1-(3,4-dichlorophenyl)-3-[3-(pyrrolidin-1-yl)propoxy]-1H-pyrazole,
1-{2-[1-(3,4-Dichlorophenyl)-5-methyl-1H-pyrazol-3-yloxy]ethyl}piperazine,
1-{2-[1-(3,4-Dichlorophenyl)-5-methyl-1H-pyrazol-3-yloxy]ethyl}pyrrolidin-3-amine,
4-{2-[1-(3,4-Dichlorophenyl)-4,5-dimethyl-1H-pyrazol-3-yloxy]ethyl}morpholine,
2-[1-(3,4-Dichlorophenyl)-4,5-dimethyl-1H-pyrazol-3-yloxy]-N,N-diethylethanamine,
1-(3,4-Dichlorophenyl)-4,5-dimethyl-3-[2-(pyrrolidin-1-yl)ethoxy]-1H-pyrazole,
1-(3,4-Dichlorophenyl)-4,5-dimethyl-3-[3-(pyrrolidin-1-yl)propoxy]-1H-pyrazole,
1-{2-[1-(3,4-Dichlorophenyl)-4,5-dimethyl-1H-pyrazol-3-yloxy]ethyl}piperidine,
4-{4-[1-(3,4-dichlorophenyl)-1H-pyrazol-3-yloxy]butyl}morpholine,
(2S,6R)-4-{4-[1-(3,4-dichlorophenyl)-1H-pyrazol-3-yloxy]butyl}-2,6-dimethylmorpholine,
1-≡4-[1-(3,4-Dichlorophenyl)-1H-pyrazol-3-yloxy]butyl)piperidine,
1-(3,4-Dichlorophenyl)-3-[4-(pyrrolidin-1-yl)butoxy]-1H-pyrazole,
4-[1-(3,4-dichlorophenyl)-1H-pyrazol-3-yloxy]-N,N-diethylbutan-1-amine,
N-benzyl-4-[1-(3,4-dichlorophenyl)-1H-pyrazol-3-yloxy]-N-methylbutan-1-amine,
4-[1-(3,4-dichlorophenyl)-1H-pyrazol-3-yloxy]-N-(2-methoxyethyl)-N-methylbutan-1-amine,
4-{4-[1-(3,4-dichlorophenyl)-1H-pyrazol-3-yloxy]butyl}thiomorpholine,
1-[1-(3,4-Dichlorophenyl)-5-methyl-3-(2-morpholinoethoxy)-1H-pyrazol-4-yl]ethanone,
1-{1-(3,4-dichlorophenyl)-5-methyl-3-[2-(pyrrolidin-1-yl)ethoxy]-1H-pyrazol-4-yl}ethanone,
1-{1-(3,4-dichlorophenyl)-5-methyl-3-[2-(piperidin-1-yl)ethoxy]-1H-pyrazol-4-yl}ethanone,
1-{1-(3,4-dichlorophenyl)-3-[2-(diethylamino)ethoxy]-5-methyl-1H-pyrazol-4-yl}ethanone,
4-{2-[5-Methyl-1-(naphthalen-2-yl)-1H-pyrazol-3-yloxy]ethyl}morpholine,
N,N-Diethyl-2-[5-methyl-1-(naphthalen-2-yl)-1H-pyrazol-3-yloxy]ethanamine,
1-(2-[5-Methyl-1-(naphthalen-2-yl)-1H-pyrazol-3-yloxy]ethyl)piperidine, and
5-Methyl-1-(naphthalen-2-yl)-3-[2-(pyrrolidin-1-yl)ethoxy]-1H-pyrazole,
or a pharmaceutically acceptable salt, isomer, prodrug or solvate thereof.

24. The synergistic combination according to claim 23, wherein the combination comprises 4-{2-[5-methyl-1-(naphthalen-2-yl)-1H-pyrazol-3-yloxy]ethyl}morpholine or a pharmaceutically acceptable salt, isomer, prodrug or solvate thereof.

25. The synergistic combination according to claim 24, wherein the combination comprises 4-{2-[5-methyl-1-(naphthalen-2-yl)-1H-pyrazol-3-yloxy]ethyl}morpholine hydrochloride.

26. The synergistic combination according to claim 17, wherein the Gabapentinoid is selected from the group consisting of Gabapentin, Pregabalin, Atagabalin, Imagabalin, DS-5565 and Gabapentin enacarbil or a pharmaceutically acceptable salt, isomer, prodrug or solvate thereof.

27. The synergistic combination according to claim 17, wherein the combination comprises 4-{2-[5-methyl-1-(naphthalen-2-yl)-1H-pyrazol-3-yloxy]ethyl}morpholine hydrochloride and Pregabalin.

28. The synergistic combination according to claim 17, wherein the combination comprises 4-{2-[5-methyl-1-(naphthalen-2-yl)-1H-pyrazol-3-yloxy]ethyl}morpholine hydrochloride and Gabapentin.

29. A method of treatment and/or prophylaxis of a patient suffering from pain, or likely to suffer pain, the method comprising administering to the patient in need of such a treatment or prophylaxis a therapeutically effective amount of a synergistic combination according to claim 17.

30. The method according to claim 29, wherein the analgesic effect of the Gabapentinoid is potentiated in the synergistic combination.

31. The method according to claim 29, wherein the pain is neuropathic pain.
